(19) United States
(12) Patent Application Publication
(10) Pub. No.: US 2004/0199113 A1
(43) Pub. Date: Oct. 7, 2004

(54) Single-use syringe

(75) Inventors: David Francis Capes, Singapore (SG); Steven Lau Choon Meng, Singapore (SG)

Correspondence Address: DAVID W. HIGHET, VP AND CHIEF IP COUNSEL, BECTON, DICKINSON AND COMPANY, 1 BECTON DRIVE, MC 110, FRANKLIN LAKES, NJ, 07417-1880, US

(73) Assignee: Becton, Dickinson and Company, Franklin Lakes, NJ

(21) Appl. No.: 10/835,848

(22) Filed: Apr. 30, 2004

Related U.S. Application Data
(63) Continuation of application No. 10/256,607, filed on Sep. 27, 2002.
(63) Continuation-in-part of application No. 10/706,795, filed on Nov. 12, 2003.
Application No. 10/706,795 is a continuation of application No. 09/941,030, filed on Aug. 28, 2001, now abandoned.
Application No. 09/941,030 is a continuation of application No. 09/274,117, filed on Mar. 23, 1999, now Pat. No. 6,361,525.
Application No. 09/274,117 is a continuation-in-part of application No. 09/249,431, filed on Feb. 12, 1999, now abandoned.
Application No. 09/249,431 is a continuation-in-part of application No. 09/124,447, filed on Jul. 29, 1998, now abandoned.

(51) Int. Cl. A61M 5/00

(52) U.S. Cl. 604/110

(57) ABSTRACT

A syringe comprising a barrel and a plunger rod having a proximal portion and a distal portion is provided. The distal portion includes a stopper slidably positioned in fluid-tight engagement with the inside surface of the barrel. The structure is provided for holding the proximal portion and the distal portion together during normal use of the syringe and breakable upon application of an additional force applied to the proximal portion. Structure is also provided for preventing the proximal portion from applying distally directed force to the distal portion after the breakable connection is broken.

### CROSS-REFERENCE TO RELATED APPLICATIONS

This application is a continuation of U.S. patent application Ser. No. 10/256,607 filed Sep. 27, 2002 and a continuation-in-part of U.S. patent application Ser. No. 10/706,795 filed Nov. 12, 2003, which is a continuation of U.S. patent application Ser. No. 09/941,030 filed Aug. 28, 2001 now abandoned, which is a continuation of U.S. patent application Ser. No. 09/274,117 filed May 14, 2001, now U.S. Pat. No. 6,361,525, which is a continuation-in-part of U.S. patent application No. 09/249,431 filed on Feb. 12, 1999, now abandoned, which is a continuation-in-part of U.S. application Ser. No. 09/124,447 filed Jul. 28, 1998, now abandoned.

### BACKGROUND OF THE INVENTION

The present invention relates to a syringe having a breakable plunger rod to prevent further use of the syringe after fluid delivery or injection.

Throughout the world the re-use of hypodermic syringe products which are intended for single-use only is instrumental in drug abuse and in the transfer of contagious diseases. Intravenous drug users who routinely share and re-use syringes are a high-risk group with respect to the AIDS virus. Also, the effects of multiple use are a major concern in some countries where the repeated use of syringe products during mass inoculation programs may be responsible for the spread of many diseases. Syringes are often recycled in developing countries without proper sterilization.

Many attempts have been made to remedy this problem. Some designs involve the inclusion of structure which will allow the destruction or defeating of the syringe function through a conscious act by the user, such as breaking a syringe or one of its components. In addition, there are single-use hypodermic syringes which become incapable of further use automatically upon delivery of the medication without any additional act on the part of the user.

### SUMMARY OF THE INVENTION

A syringe comprises a barrel having a fluid chamber, a proximal end, a distal end, and an elongate tip extending from the distal end having a passageway therethrough in fluid communication with the chamber, and a plunger rod. The plunger rod has a longitudinal axis, a proximal portion and a distal portion connected by a breakable connection. One of the proximal portion and the distal portion includes an axial projection having at least one transverse protuberance projecting therefrom. The protuberance is connected to the other of the proximal portion and the distal portion. The breakable connection is on the protuberance. The distal portion includes a stopper slidably positioned in fluid-tight engagement with an inside surface of the chamber for drawing fluid into and out of the chamber by movement of the plunger rod relative to the barrel. The breakable connection is strong enough to hold the proximal portion and the distal portion together during normal use of the syringe and breakable upon application of an additional force applied to the proximal portion along the longitudinal axis. Structure is provided for preventing the proximal portion from applying a distally directed force to the distal portion after the breakable connection is broken. This structure may include a projection on the proximal portion of the plunger rod configured to contact the barrel after the breakable connection is broken. The plunger rod may have a plurality of outwardly projecting ribs wherein one or all of the ribs contains a projection to limit the distal motion of the proximal portion of the plunger rod. The projection may be integral or separately attached to the plunger or barrel. The projection may also take the form of a transverse flange on the plunger rod. The plunger rod may contain a distally directed extension on the distal portion shaped to fit within the passageway of the elongate tip of the syringe barrel for displacing fluid from the passageway when the plunger is positioned distally with respect to the barrel.

### BRIEF DESCRIPTION OF THE DRAWINGS

FIG. 1 is an exploded perspective view of the plunger rod and barrel of a syringe of the present invention.

FIG. 2 is an enlarged perspective view of the plunger rod of the syringe of the present invention having a distal portion and a proximal portion connected by a breakable connection.

FIG. 3 is a partial cross-sectional side elevation view of a syringe assembly using the plunger rod of FIG. 2.

FIG. 4 is a side elevational view of the syringe of FIG. 4 illustrated with a force being applied to break the connection between the proximal and distal portions of the plunger rod.

FIG. 5 is a side elevational view of the syringe of FIG. 4 illustrating the separated proximal and distal ends of the plunger rod.

FIG. 6 is a side elevational view of the plunger rod.

FIG. 7 is a top plan view of the plunger rod of FIG. 6.

FIG. 8 is a perspective view of an alternative embodiment of the plunger rod of the present invention.

FIG. 9 is an enlarged partial perspective view of the plunger rod of FIG. 8.

FIG. 10 is a partial cross-sectional view of the plunger rod of FIG. 8 in a syringe barrel.

FIG. 11 is a partial cross-sectional view of another alternative embodiment of the syringe of the present invention.

FIG. 12 is another alternative embodiment of the plunger rod of the present invention.

### DETAILED DESCRIPTION

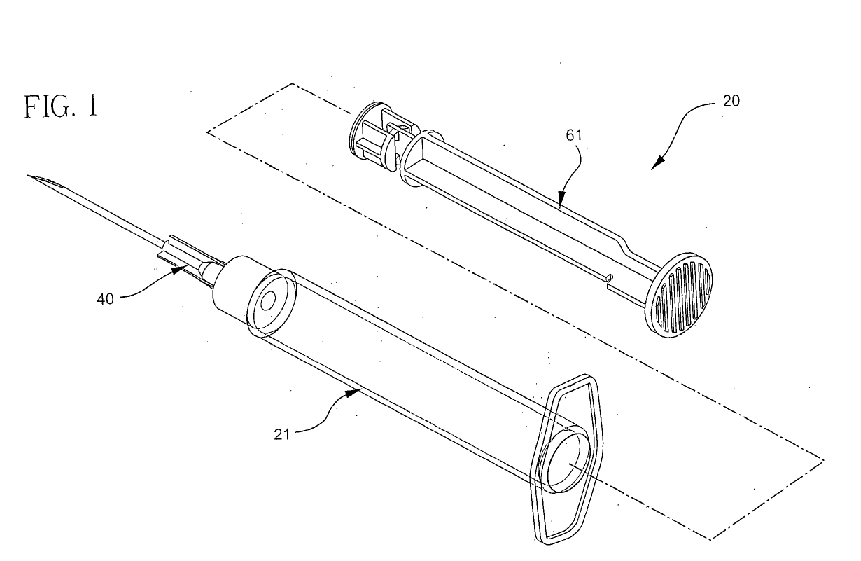

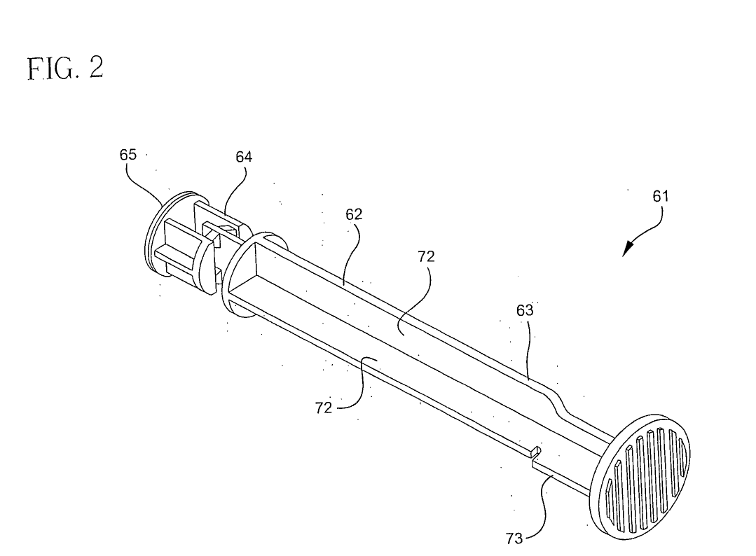

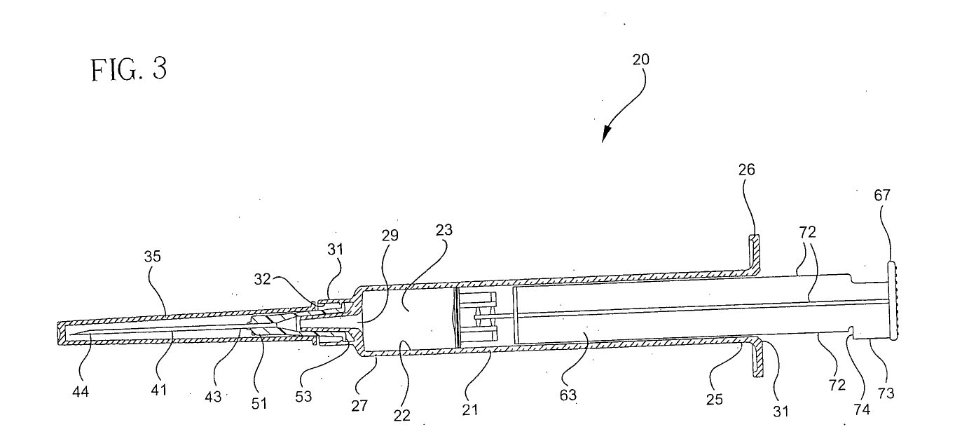

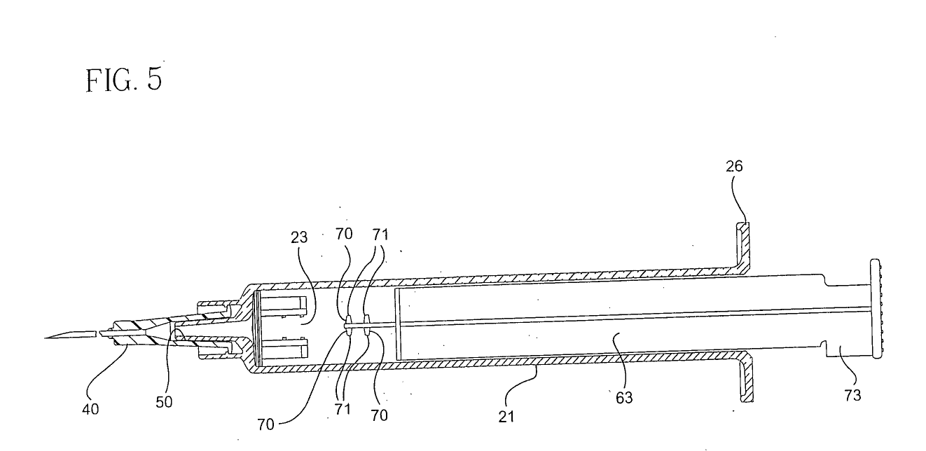

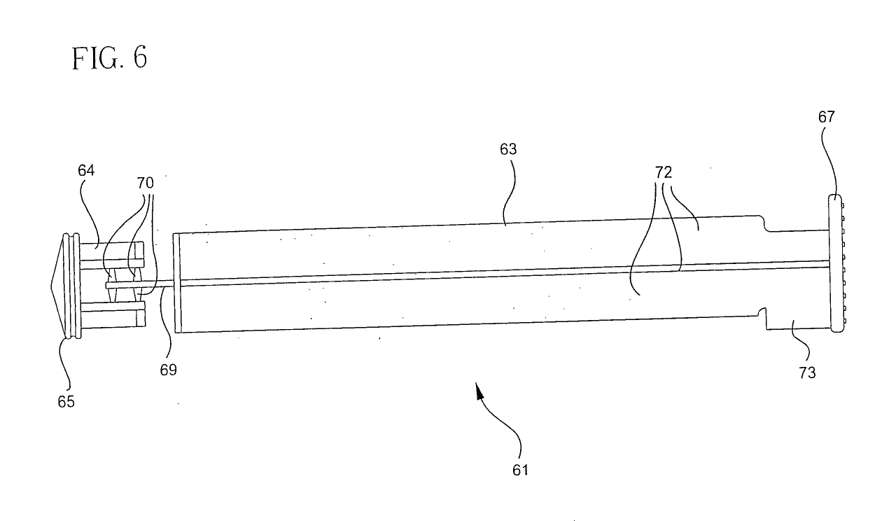

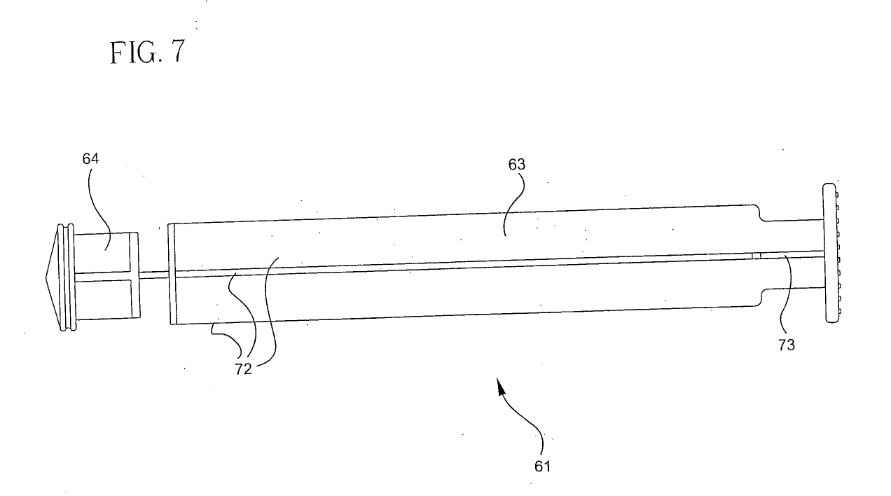

Adverting to FIGS. 1-7 a syringe 20 includes a barrel 21 having a barrel inside surface 22, a fluid chamber 23, a proximal end 25, a distal end 27 and an elongate tip 28 extending from the distal end having a passageway 29 therethrough in fluid communication with the chamber. In this embodiment the elongate tip is preferably frusto-conically shaped.

Barrel 21 preferably includes a collar 31 having an inside surface 32 and at least one third to engage a needle hub.

The syringe of the present invention is intended to be used with a needle assembly 40 including a cannula 41 having a proximal end 43 a distal end 44 and a lumen 45 therethrough. The needle assembly also includes a hub 47 having an open proximal end 49 with a cavity 50 therein, and a distal end 51 joined to proximal end 43 of cannula 41 so that lumen 45 is in fluid communication with cavity 50. A removable needle shield 35 is provided to protect the cannula before use. It is within the purview of the present invention to include needle assemblies having one-piece construction wherein the cannula and the hub are formed of one piece.

Syringe barrel 22 preferably includes a collar 31 around the elongate tip having a thread on the inside surface of the collar. This configuration is often referred to as a locking luer collar. The needle assembly with the hub having outwardly extending projections 53 is placed on the distal end of the syringe by aligning the distal tip of the syringe with a cavity in the hub and moving the needle assembly toward the syringe so that the outward projections of the hub engage the thread. The needle assembly is then rotated or screwed into the locking luer collar so that the needle assembly is held tightly on the distal tip of the syringe barrel through interaction of the locking luer collar thread and the projections on the hub. This is an excellent structure for most applications since it allows for applying the appropriate sized needle assembly at the time of use and for changing needle assemblies during a procedure which may require two or more different sizes.

The syringe of the present embodiment includes a plunger rod 61 having an elongate body portion 62 defining longitudinal axis 66 and having a proximal portion 63, a distal portion 64 and a stopper 65 on the distal portion. The stopper is slidably positioned in fluid-tight engagement with the barrel inside surface of the chamber for drawing fluid in and out of the chamber by movement of the plunger rod relative to the barrel. The plunger rod extends outwardly from proximal end 25 of the barrel. The plunger rod is accessible outside of proximal end 25 of the barrel and is provided to move the stopper along the barrel to force fluid into and out of chamber 23 through passageway 29. Disc-shaped plunger rod flange 67 is provided as a convenient structure for applying forces to move the plunger rod with respect to barrel 21. A flange 26 is also provided at the proximal end of the barrel to facilitate handling and for maintaining the relative position of the barrel with respect to the plunger rod during fluid transfer using known procedures.

It is within the purview of the present invention to include plunger rods and stoppers which are separately formed or integrally formed of the same material or different materials such as in two-color molding, or separately formed of the same or different materials and joined together by mechanical means, adhesives, ultrasonic welding, heat sealing or other suitable means. Stoppers are preferably made of elastomeric material such as natural rubber, synthetic rubber, thermoplastic elastomers and combinations thereof. It is understood that the plunger of the present embodiment merely illustrates these many possibilities.

Proximal portion 63 and distal portion 64 of the plunger rod are connected by a breakable connection 68. Breakable connection 68 is strong enough to hold proximal portion 63 and distal portion 64 together during normal use of the syringe and is breakable upon application of additional force to the proximal portion.

In this embodiment, there are a one or more breakable connections connecting proximal portion 63 and distal portion 64. Specifically, proximal portion 63 includes a distal projection 69 having at least one transverse protuberance projecting therefrom. In this preferred embodiment, there are four transverse protuberances 70. The protuberances are connected to distal portion 64 and the breakable connection is on the protuberance. In this embodiment breakable connection 71 is preferably on the distal end of the protuberance. The distal projection may be circularly shaped and fit into a cylindrically shaped recess in the distal portion. With this structure a single protuberance extending up to 360 degrees may be used. The structure may also be reversed so that the projection extends proximally from the distal portion toward the proximal portion.

In this preferred embodiment, proximal portion 63 and distal portion 64 and the breakable connections are integrally molded of plastic material. A wide variety of plastic materials are suitable for molding the plunger with polystyrene, polypropylene and polyethylene being preferred. It is important to control the modulus of elasticity of material selected for the transverse protuberances which are part of the breakable connection between the proximal portion and the distal portion of the plunger rod. The breakable connection must break or fail before the proximal portion of the plunger contacts the distal portion of the plunger. If the modulus is too high the break will occur too easily, causing premature breakage. If the modulus is too low the breakable connection may not break because the proximal portion and the distal portion will contact each other to resist further relative movement between the proximal and distal portions. Also, careful controlling of the modulus will allow use of materials such as polypropylene which would not normally be used to form a breakable connection. It is preferred to have a modulus of elasticity be within the range of about 800 MPa to 4000 MPa.

The breakable connections can be anywhere along the protuberance, at its proximal end, its distal end, or in between, depending on, among other things, the geometry of the protuberance. A breakable connection can also be made by connecting the protuberance to the distal end or to the proximal end using a frangible adhesive. The protuberance may be very short and made entirely of adhesive or frangible material. The connection can also be made using a shear pin passing through the distal projection 69 and distal portion 64. The shear pin may be made of plastic with one or more notches or stress risers suitably placed to cause breaking at the desired force levels. The breakable connection between the proximal portion and the distal portion may also be accomplished by using a snap-fit arrangement a portion of which is damaged or broken when the desired force is applied. In this latter situation, the distal portion and the proximal portion can be individually molded and snapped together during the assembly process.

In use, the syringe of this embodiment can be filled from a vial, ampoule or other suitable container using known safe procedures. An important advantage of the present embodiment is that the plunger can be moved back and forth along the barrel as many times as necessary to properly fill the syringe barrel. For example, the syringe barrel may be filled with sterile water and then the sterile water can be injected into a vial containing a lyophilized medication which is then drawn back into the syringe barrel. Many single-use syringes in the prior art only allow one proximal motion of the plunger with respect to the barrel. With these single-use syringes, once the plunger is moved in a distal direction with respect to the barrel it can no longer be withdrawn. Therefore, mixing sterile water and a lyophilized medication as described above is not possible.

Another advantage of the present embodiment is that the plunger can be moved to its distal-most position with respect to the barrel and then moved proximally. Some prior art single use syringes automatically lock the plunger to the barrel when the plunger is moved to its distal-most position. These prior art designs can lead to unintentional locking of the plunger before use and can compromise filling and mixing procedures.

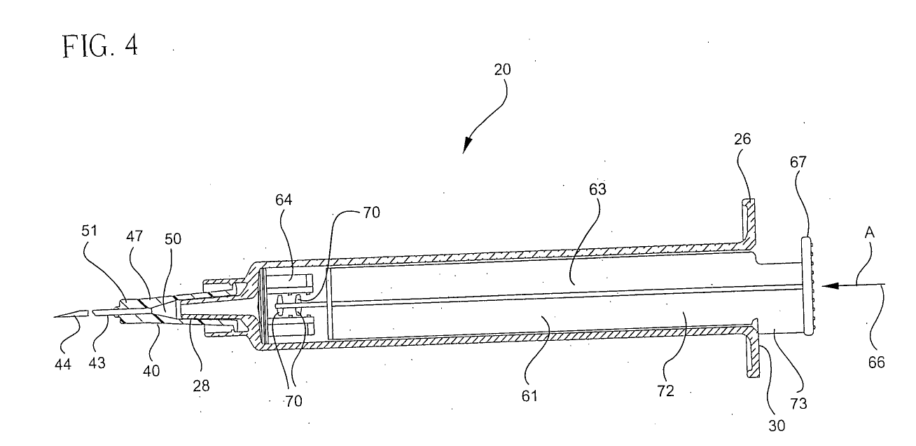

The liquid in the barrel can now be injected into a patient or delivered in another suitable manner such as through the pierceable septum of a catheter connector. Upon completion of the injection the user can apply an additional force indicated as A in FIG. 4, to the proximal portion. In this embodiment the breakable connections are broken by the application of a distally directed force A applied to the proximal end of the proximal portion along longitudinal axis 66. Force A is sufficient to break the breakable connections: which causes the plunger rod to separate into two or more unusable pieces. In this enablement the proximal portion and the distal portion are separated as illustrated in FIG. 4.

After breaking the breakable connection the proximal portion of the plunger rod moves distally at high speed. If the proximal portion is permitted to impact the distal portion at this time, the distal portion may compress whatever fluid remains in the dead space between the plunger rod and the roof of the barrel. This action can cause some fluid in the dead space to be squeezed out of the passageway of the elongate tip at high speed, resulting in spraying some of the fluid from the nozzle. An important advantage of the present invention is that the syringe includes means for preventing proximal portion 63 of the plunger rod from applying distally directed force to distal portion 64 after the breakable connection is broken. Preferred means for preventing contact and thus the application of distally directed force by the proximal portion to the distal portion after the breakable connection is broken include providing a projection on the proximal portion configured to contact some portion of the barrel after the connection is broken and before contact with the distal portion can be made. In this preferred embodiment, the plunger rod 61 includes a plurality of outwardly projecting ribs 72 and a projection 73 on at least one of ribs 72. The projection is configured to contact some portion of the barrel after the breakable connection is broken and before the proximal portion can contact the distal portion of the plunger rod in order to prevent unwanted discharge of any residual contents of the syringe chamber. In this embodiment, projection 73 has a leading edge 74 which contacts proximal surface 30 on the barrel. It is understood that any combination of projections and/or recesses on the plunger rod which it can interact with recesses and/or projections on the barrel to achieve the same result are within the purview of the present invention and that the structure illustrated in this preferred embodiment is merely representative of these many possibilities. In this embodiment, projection 73 is on one of ribs 72, however, a projection can be on one or more of the ribs provided.

An advantage of the present invention is that the breakable connection can be broken using any one-handed technique, for example, pressing on plunger rod flange 67 in direction A with the thumb of one hand while holding the syringe barrel and/or the syringe barrel flange with some or all of the remaining fingers. This is desirable over a two-handed technique wherein the barrel must be held by one hand and the plunger by another to carry out a breaking manipulation such as bending or twisting.

The embodiment of FIGS. 1-7 is a significant advance over single-use syringes of the prior art. In particular, it allows multiple strokes of the plunger with respect to the barrel without automatically locking and rendering the syringe unusable. This design also allows the plunger to move to its distal-most position inside the barrel without automatically locking the plunger to the barrel. It also provides a mechanism to prevent or discourage re-use. The plunger is breakable so that its proximal and distal ends are separated and the syringe, can no longer be used to inject medication. Structure is provided to prevent unwanted force on the distal portion of the plunger rod after the breakable connection is broken in order to prevent unwanted discharge of residual medication from the chamber and/or passageway. Further, the plunger breaking feature is accomplished by a simple one-handed procedure.

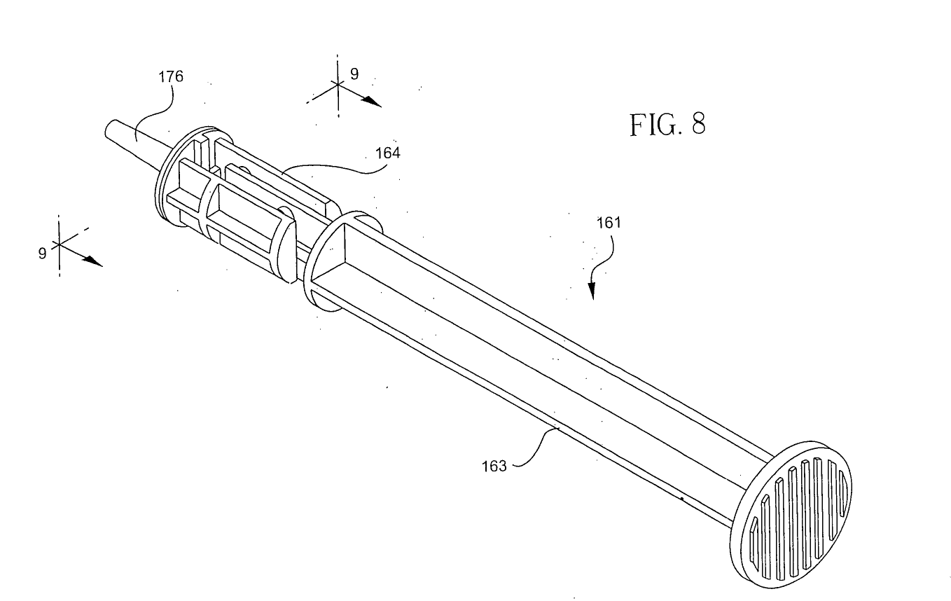

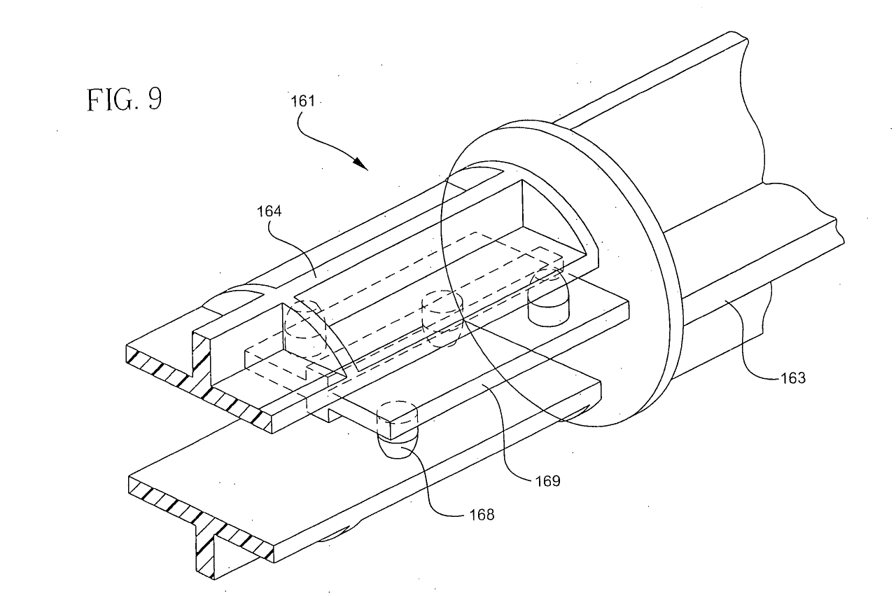

FIGS. 8-10 illustrate an alternative embodiment of the present invention. In this embodiment, a syringe 120 comprising a barrel 121 having a fluid chamber 123, a proximal end 125, a distal end 127 and an elongate tip 128 extending from the distal end and having a passageway 129 therethrough in fluid communication with the chamber.

A plunger rod 161 having a longitudinal axis 166, a proximal portion 163 and a distal portion 164 connected by a breakable connection 168. It is preferred that either proximal portion 163 or distal portion 164 include an axial projection. In this embodiment an axial projection 169 is on proximal portion 163. Axial projection 169 includes at least one transverse protuberance projecting therefrom. In this embodiment, there are four transverse protuberances 170. The protuberances are connected to distal portion 164 and the breakable connection is on the protuberances. The transverse protuberances project from opposite sides of the axial projection. Also, in this embodiment, two of transverse protuberances 170 project in substantially the opposite direction from the other two transverse protuberances 170. It is preferable that proximal portion 163 and distal portion 164 and the transverse protuberances 170 which include breakable connections are integrally molded of plastic material.

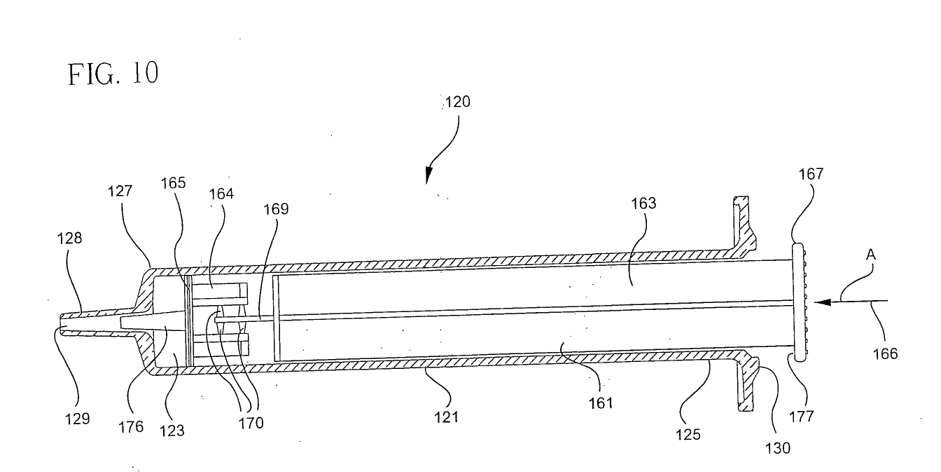

Plunger rod 161 in this embodiment functions similarly to plunger rod 61 in the embodiments of FIGS. 1-7. Distal portion 164 includes a stopper 165 slidably positioned in fluid-tight engagement with an inside surface of chamber 123 for drawing fluid into and out of the chamber by movement of plunger rod 161 relative to barrel 121. The breakable connection is strong enough to hold the proximal portion and the distal portion together during normal use of the syringe and breakable upon application of an additional force applied to the proximal portion along the longitudinal axis such as distally directed force A in FIG. 10. It is preferred that the additional distally directed force required to break the breakable connection be within the range of about 2.2 kg to 6.8 kg (5 lbs. to 15 lbs.). The proper force depends on various dimensions of the syringe barrel and plunger, the viscosity of the liquid being delivered and the mechanical and hydraulic forces encountered by the filling and delivery process. If the breakable connection is too weak the proximal and distal portions will separate during normal use of the syringe and if the force required to break the breakable connection is too high the user may not be able to easily break the breakable connection as intended. In this embodiment axial projection 169 is planar shaped having two opposed side walls with at least one transverse protuberance projecting from each of the side walls. In this embodiment, there are two transverse protuberances projecting from each side wall. This structure because of the planar axial projection and the diagonal positioning of the transverse protuberances is preferred because it creates a secure linkage between the proximal portion and the distal portion and it is strong with respect to rotational movement or rotational forces applied to the plunger and weaker with respect to axial forces applied to the plunger so that rotational forces should not break the breakable connection.

In this embodiment, proximal portion 163 is prevented from applying distally directed force to distal portion 164 after the breakable connection is broken by action of thumb press 167 on the proximal portion contacting barrel 121. In particular, a distal surface 174 on the thumb press contacts proximally directed projection 130 on the barrel to prevent unwanted contact with the distal portion.

This embodiment further includes distally directed projection 176 on the distal portion for displacing fluid in passageway 129 when the plunger is positioned distally with respect to the barrel. This is an important feature of this embodiment because it saves medication that might otherwise remain in the passageway after the injection process was completed. In an immunization program involving millions of syringes, the amount of medication saved by the cooperative relationship between the distally directed projection and the passageway can be substantial. Even a four percent improvement can result in a free dose for every twenty-five syringes used.

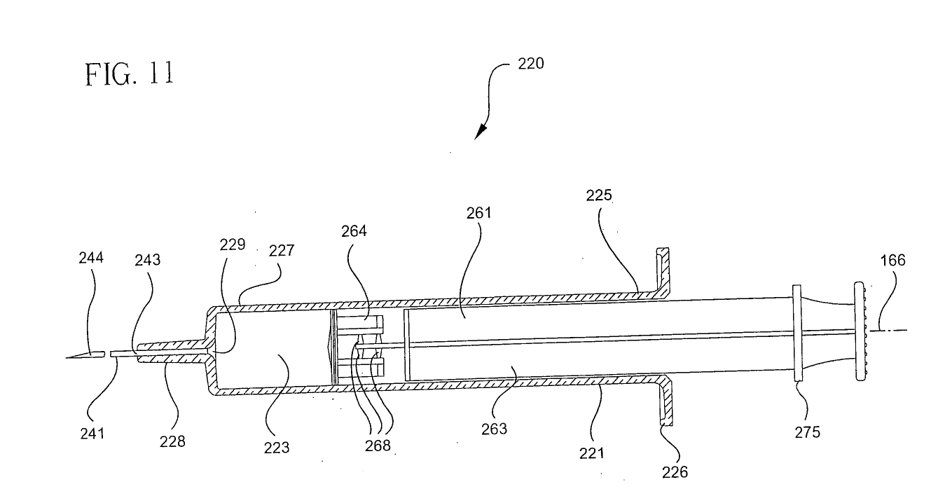

FIG. 11 illustrates another alternative embodiment of the present invention. This embodiment includes a syringe 220 which functions similarly to the syringe of the embodiment of FIGS. 1-7. The syringe includes a barrel 221 having a fluid chamber 223, a proximal end 225, a distal end 227 and an elongate tip 228 extending from the distal end and having a passageway 229 therethrough in fluid communication with the chamber. The syringe further includes a cannula 241 having a proximal end 243, a distal end 244 and a lumen therethrough. The proximal end of the cannula is joined to elongate tip 228 so that the lumen is in fluid communication with passageway 229. A plunger rod 261, a proximal portion 263 and a distal portion 264 connected by a breakable connection 268.

Means for preventing proximal portion 263 from applying a distally directed force to distal portion 264 after the breakable connection is broken is provided by action between intermediate flange 275 on plunger rod 261 and barrel 221. In particular, forward motion of the proximal portion of the plunger rod is limited by contact between intermediate flange 275 and barrel flange 226 to prevent unwanted discharge of residual fluid in the chamber and/or passageway.

Cannula 241 is preferably permanently connected to the barrel so that it is not replaceable or removable during normal use. There are many ways to connect the cannula to a barrel including adhesives, such as epoxy. An advantage of this embodiment is that it can be configured to save medication from being wasted and, more importantly, when the breakable connection is broken the plunger is no longer usable and the needle cannot be removed from the syringe to be used again in an undesirable manner.

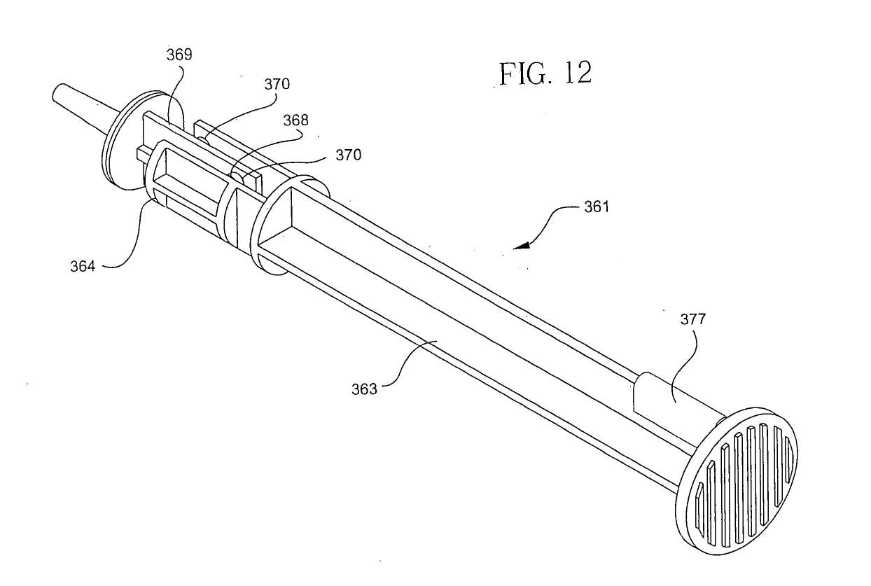

FIG. 12 illustrates an alternative elongate plunger rod 361, having a proximal portion 363 and a distal portion 364 connected by a breakable connection 368. Either the proximal portion or the distal portion includes an axial projection 369 having at least one traverse protuberance 370 projecting therefrom. In this embodiment, the axial projection is part of distal portion 364 and the protuberance is connected to the proximal portion. The breakable connection is on the protuberance and in this embodiment it is generally in the area where the transverse protuberance joins the proximal portion. Although the plunger in the embodiment of FIG. 12 functions similarly to the plunger in the embodiment of FIGS. 1-7, the axial projection in this embodiment is on the distal portion while the axial projection in the embodiment of FIGS. 1-7 is on the proximal portion. Accordingly, either the proximal portion or the distal portion may include the axial projection having at least one transverse protuberance. In this embodiment there are four transverse protuberances, two of which project from one side of the axial projection and the other two transverse protuberances project from the other side of the axial projection. Means for preventing proximal portion 363 from contacting distal portion 364 after the breakable connection is broken is provided by interaction between clip on projection 377 which will contact the barrel before the proximal portion of the plunger rod can contact the distal portion of the plunger rod to prevent unwanted discharge of any residual fluid in a chamber and/or passageway. The clip-on projection can be installed after the plunger rod is formed and be made of metal or plastic or any suitable material which will adequately prevent forward motion of the plunger rod upon contact with the barrel. Projection may be made of stainless steel sheet metal having internal teeth to engage the plunger rod structure and hold it in a fixed position with respect to the proximal portion.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A syringe comprising:
a barrel having a fluid chamber, a proximal end, a distal end and an elongate tip extending from said distal end having a passageway therethrough in fluid communication with said chamber;
a plunger rod having a longitudinal axis, a proximal portion and a distal portion connected by a breakable connection, one of said proximal portion and said distal portion including an axial projection having at least one transverse protuberance projecting therefrom, said protuberance being connected to the other of said proximal portion and said distal portion, said breakable connection being on said protuberance, said distal portion including a stopper slidably positioned in fluid-tight engagement with an inside surface of said chamber for drawing fluid into and out of said chamber by movement of said plunger relative to said barrel, said breakable connection being strong enough to hold said proximal portion and said distal portion together during normal use of said syringe and breakable upon application of an additional force applied to said proximal portion along said longitudinal axis; and
means for preventing said proximal portion from applying a distally directed force to said distal portion after said breakable connection is broken.

2. The syringe of claim 1 wherein said means for preventing includes a projection on said proximal portion configured to contact said barrel after said breakable connection is broken.

3. The syringe of claim 2 wherein said proximal portion includes a plurality of outwardly projecting ribs and said projection is on at lest one of said ribs.

4. The syringe of claim 3 wherein said projection is integrally formed with said rib.

5. The syringe of claim 2 wherein said projection is a transverse flange on said proximal portion.

6. The syringe of claim 5 wherein said flange is on the proximal end of said proximal portion.

7. The syringe of claim 1 wherein said axial projection includes a plurality of transverse protuberances.

8. The syringe of claim 7 wherein said transverse protuberances project from opposite sides of said axial projection.

9. The syringe of claim 8 wherein said axial projection includes four transverse protuberances, two of said transverse protuberances projecting in substantially the opposite direction from the other two of said transverse protuberances.

10. The syringe of claim 1 wherein said axial projection is planar shaped having two opposed side walls and at least one transverse protuberance projecting from each of said two side walls.

11. The syringe of claim 1 further including a distally directed extension shaped to fit within said passageway for displacing fluid from said passageway when said plunger is positioned distally with respect to said barrel.

12. The syringe of claim 1 wherein said proximal portion, said distal portion and said breakable connection are integrally molded of plastic material.

13. The syringe of claim 1 wherein said breakable connection is made of material selected from the group of polyethylene, polystyrene, polypropylene and adhesives.

14. The syringe of claim 1 wherein said stopper and said distal portion are integrally molded of plastic material.

15. The syringe of claim 1 further including a needle assembly including a cannula having a proximal end, a distal end and a lumen therethrough, a hub having an open proximal end with a cavity therein, and a distal end joined to said proximal end of said cannula so that said lumen is in fluid communication with said cavity, said needle assembly being connected to said barrel so that said elongate tip of said barrel is in said cavity of said hub.

16. The syringe of claim 1 further including a cannula having a proximal end, a distal end and a lumen therethrough, said proximal end of said cannula being joined to elongate tip so that said lumen is in fluid communication with said passageway.

17. The syringe of claim 1 further including a cannula having a proximal end, a distal end and a lumen therethrough, said proximal end of said cannula being permanently attached to said elongate tip so that said lumen is in fluid communication with said passageway.

18. The syringe of claim 17, wherein said distal end is selected from the group consisting of a sharpened tip or a blunt tip.

19. A syringe comprising:
a barrel having a fluid chamber, a proximal end, a distal end and an elongate tip extending from said distal end having a passageway therethrough in fluid communication with said chamber;
a plunger rod having a longitudinal axis, a proximal portion and a distal portion, said distal portion including a stopper slidably positioned in fluid-tight engagement with an inside surface of said chamber for drawing fluid into and out of said chamber by movement of said plunger relative to said barrel;
means for holding said proximal portion and said distal portion together during normal use of said syringe and breakable upon application of an additional force applied to said proximal portion along said longitudinal axis; and
means for preventing said proximal portion from applying a distally directed force to said distal portion after said breakable connection is broken.

20. The syringe of claim 19 wherein said means for preventing includes a projection on said proximal portion configured to contact said barrel after said breakable connection is broken.

21. The syringe of claim 20 wherein said proximal portion includes a plurality of outwardly projecting ribs and said projection is on at least one of said ribs.

22. The syringe of claim 21 wherein said projection is integrally formed with said rib.

23. The syringe of claim 20 wherein said projection is a transverse flange on said proximal portion.

24. The syringe of claim 23 wherein said flange is on the proximal end of said proximal portion.

25. The syringe of claim 19 wherein said additional force is a distally directed force within the range of about 2.2 kg to 6.8 kg (5 pounds to 15 pounds).

26. The syringe of claim 19 further including a distally directed extension on said distal portion shaped to fit within the passageway of an elongate tip of said syringe barrel for displacing fluid from said passageway when said plunger is positioned distally with respect to said barrel.

27. The syringe of claim 19 further including a needle assembly including a cannula having a proximal end, a distal end and a lumen therethrough, a hub having an open proximal end with a cavity therein and a distal end joined to said proximal end of said cannula so that said lumen is in fluid communication with said cavity, said needle assembly being connected to said barrel so that said elongated tip of said barrel is in said cavity of said hub.

28. The syringe of claim 27, wherein said needle assembly is permanently connected to said barrel.

29. The syringe of claim 28, wherein said distal end has a shape selected from the group consisting of a sharpened tip and a blunt tip.

30. A syringe comprising:
a barrel having a fluid chamber, a proximal end, a distal end and an elongated tip extending from said distal end having a passageway therethrough in fluid communication with said chamber, a collar surrounding said tip, and at least one deflectable locking tab projecting radially inwardly from said collar;
a needle assembly including a cannula having a proximal end, a distal end and a lumen therethrough, said proximal end of said cannula being permanently attached to said elongate tip so that said lumen is in fluid communication with said passageway; and
a plunger including an elongated plunger rod having a longitudinal axis, a proximal portion and a distal portion connected by a breakable connection, said distal portion including a stopper slidably positioned in fluid-tight engagement with an inside surface of said chamber for drawing fluid into and out of said chamber by movement of said plunger relative to said barrel, said breakable connection being strong enough to hold said proximal portion and said distal portion together during normal use of said syringe and breakable upon application of additional force to said proximal portion.

31. The syringe of claim 30, wherein said distal end has a shape selected from the group consisting of a sharpened tip and a blunt tip.
